(19) United States
(12) Patent Application Publication
(10) Pub. No.: US 2021/0169351 A1
(43) Pub. Date: Jun. 10, 2021

(54) HEALTHCARE DEVICE AND HEALTHCARE SYSTEM USING SAME

(71) Applicant: ONESOFTDIGM CO., LTD, Gyeongsangbuk-do (KR)

(72) Inventor: Dae Ho LEE, Gyeongsangbuk-do (KR)

(21) Appl. No.: 16/760,442

(22) PCT Filed: Oct. 1, 2018
(86) PCT No.: PCT/KR2018/011659

§ 371 Date: Jun. 5, 2020

(30) Foreign Application Priority Data
Nov. 1, 2017 (KR) 10-2017-0144886

(51) Int. Cl. A61B 5/026; A61B 5/00; A61B 5/11; G16H 50/30; G16H 40/63; G16H 20/30

(52) U.S. Cl. CPC A61B 5/0261; A61B 5/4884; A61B 5/1123; A61B 2562/0219; G16H 40/63; G16H 20/30; G16H 50/30

(57) ABSTRACT

A health care device and a health care system using the same are provided. The health care device and the health care system using the same include a terminal unit including a health care device including a data measurement unit, a calculation unit, an illumination unit, a power supply unit, and a communication unit, and a service providing unit, so that precise biometric data measurement is possible, and based on measured biometric data, a high performance and high utilization health care device that provides services such as measurement information history management, exercise recommendation, diet management, and body analysis, and a health care system using the same may be provided.

### TECHNICAL FIELD

The present invention relates to a health care device and a health care system using the same, and more particularly, to a health care device collecting biometric data from a user and a health care system using the same.

### BACKGROUND ART

Due to the aging of the population, the increase in income and the spread of the wellness culture, the medical paradigm is shifting from treatment to prevention and management.

Accordingly, the health care industry is drawing attention worldwide, and recently, the field of smart health care, in which medical and information & communication technology (ICT) is converged, is expanding.

The smart health care field is being used in various fields such as sports, daily life, beyond medical services, and recently, research on a health care system combined with a wearable device has been conducted for reliable and convenient data collection.

In addition, the existing health care system is provided in a form in which a communication function is combined with a personal medical device, and thus performs only a user's rapid bio-data measurement and calculation function. Accordingly, since expertise is required to analyze the calculated result data, there are cases where the majority of users stop using without benefit or assistance.

### DESCRIPTION OF EMBODIMENTS

### Technical Problem

An object of the present invention for solving the above problems is to provide a health care device capable of quickly and accurately collecting biometric data and a health care system using the same.

Another object of the present invention for solving the above problems is to provide a high-performance health care device that is easy to analyze the result data and a health care system using the same.

Another object of the present invention for solving the above problems is to provide a highly utilized health care device capable of interworking with external services and a health care system using the same.

Another object of the present invention for solving the above problems is to provide a high-efficiency health care device having no place restrictions and a health care system using the same.

### Solution to Problem

According to an embodiment of the present invention for achieving the above object, a health care device includes: a data measurement unit configured to measure basic data from a sensor unit including an illuminance sensor and an acceleration sensor and a circuit unit measuring electrical resistance; and a calculation unit configured to calculate biometric data of a user from the basic data.

According to an embodiment of the present invention for achieving the above object, the health care device may further include an illumination unit for irradiating light to at least a portion of the user's body, wherein the illuminance sensor may measure an amount of transmission of light irradiated from the illumination unit to obtain blood flow change data.

According to an embodiment of the present invention for achieving the above object, the calculation unit may include: a first calculation unit configured to calculate heart rate data from the blood flow change data obtained from the illuminance sensor; a second calculation unit configured to calculate a stress index from the blood flow change data; a third calculation unit configured to calculate activity data from walking data measured by the acceleration sensor; and a fourth calculation unit configured to calculate a body composition from electrical resistance data measured by the circuit unit.

According to an embodiment of the present invention for achieving the above object, the first calculation unit may extract a specific pattern from the blood flow change data and calculate a period for the pattern to measure heart rate data.

According to an embodiment of the present invention for achieving the above object, the second calculation unit may calculate a stress index according to a change in peak interval of a graph generated by secondary differentiation of the blood flow change data.

According to an embodiment of the present invention for achieving the above object, the calculation unit may further include a fifth calculation unit that calculates obesity by the stress index, the activity data, and the body fat mass.

According to another embodiment of the present invention for achieving the above object, a health care system includes: a health care device including a calculation unit for calculating biometric data of a user; and a terminal including a service providing unit for analyzing the biometric data received from the health care device.

According to another embodiment of the present invention for achieving the above object, the service providing unit of the health care system may include a first service providing unit for classifying at least one biometric data for each measurement session and displaying the classified biometric data in a trend graph form to provide a measurement information history management service.

According to another embodiment of the present invention for achieving the above object, the trend graph of the health care system may be selectively displayed by a user.

According to another embodiment of the present invention for achieving the above object, the service providing unit of the health care system may include a second service providing unit for calculating a target heart rate (Beats Per Minute (BPM)) using at least one of the biometric data and providing an exercise recommendation service.

According to another embodiment of the present invention for achieving the above object, a calculation method for the target heart rate (Beats Per Minute (BPM)) of the health care system may vary according to a user's exercise skill.

According to another embodiment of the present invention for achieving the above object, in the case of a beginner with low exercise skill, the health care system may calculate the target heart rate (Beats Per Minute (BPM)) by using a user's age and exercise intensity.

According to another embodiment of the present invention for achieving the above object, in the case of an expert with high exercise skill, the health care system may calculate the target heart rate (Beats Per Minute (BPM)) by using a maximum heart rate, a stable heart rate, and an exercise intensity.

According to another embodiment of the present invention for achieving the above object, the service providing unit of the health care system may include a third service providing unit for providing a diet management service by calculating an intakeable calorie amount from at least one of the biometric data.

According to another embodiment of the present invention for achieving the above object, the service providing unit of the health care system may include a fourth service providing unit for providing a body type analysis service by calculating a recommended daily calorie amount from user's weight information and daily intake calorie amount.

According to another embodiment of the present invention for achieving the above object, at least one biometric data measured by the health care device of the health care system may be transmitted to the terminal by Bluetooth communication.

### Advantageous Effects of Disclosure

The health care device according to the embodiment of the present invention and the health care system may provide a health care device capable of collecting high-performance bio-data and a health care system using the same by inducing biometric data input from the user through a messenger server providing a chatbot service.

In addition, the health care device and the health care system using the same according to an embodiment of the present invention may provide a high-efficiency health care device for easily extracting biometric data from a text message input from a user by a keyword extraction unit and a health care system using the same.

In addition, the health care device and the health care system using the same according to an embodiment of the present invention may provide a high-efficiency health care device for easily collecting biometric data and a health care system using the same by a messenger server interworking with an external server.

In addition, the health care device and the health care system using the same according to an embodiment of the present invention may provide a highly utilized health care service that can be used without restriction of a place by using a cloud server based data storage unit, a data analysis unit, and a data processing unit.

In addition, since the health care device according to the embodiment of the present invention and the health care system using the same are capable of collecting biological data by a measuring device, a high-precision health care device and a health care system using the same may be provided.

In addition, the health care device and the health care system using the same according to an embodiment of the present invention may provide a high-efficiency health care device capable of providing various analysis services and a health care system using the same through a data processing unit interworking with an external server.

### BRIEF DESCRIPTION OF DRAWINGS

FIG. 1 is a configuration diagram for explaining a health care device according to an embodiment of the present invention.

FIG. 2 is a conceptual diagram illustrating a health care device according to an embodiment of the present invention.

FIG. 3 is a graph for explaining a health care device according to an embodiment of the present invention.

FIG. 4 is a perspective view for explaining a health care system according to an embodiment of the present invention.

FIG. 5 is a block diagram illustrating a health care system according to an embodiment of the present invention.

FIG. 6 is a graph for explaining a health care system according to an embodiment of the present invention.

FIG. 7 is a graph for explaining a health care system according to an embodiment of the present invention.

FIG. 8 is an image for explaining a health care system according to an embodiment of the present invention.

### MODE OF DISCLOSURE

The present invention can be applied to various changes and can have various embodiments, and specific embodiments will be illustrated in the drawings and described in detail. However, this does not limit various embodiments of the present invention to a specific embodiment and it should be understood that the present invention covers all the modifications, equivalents, and/or replacements of this disclosure provided they come within the scope of the appended claims and their equivalents. Like reference numerals refer to like elements throughout the drawings.

It will be understood that the terms “first” and “second” are used herein to describe various components but these components should not be limited by these terms. The above terms are used only to distinguish one component from another. For example, a first component may be referred to as a second component and vice versa without departing from the scope of the present invention. The term “and/or” includes a combination or any one of a plurality of listed items.

When it is mentioned that a certain component is “coupled with” or “connected with” another component, it should be understood that the certain component is directly “coupled with” or “connected with” to the other component or a further component may be located therebetween. In contrast, when it is mentioned that a certain component is “directly coupled with” or “directly connected with” another component, it will be understood that a further component is not located therebetween.

Terms used in this specification are used to describe specific embodiments, and are not intended to limit the scope of the present invention. The terms of a singular form may include plural forms unless otherwise specified. In this specification, the term “include” or “have” specifies the existence of a feature, a number, a step, an operation, a component, a part, or a combination thereof listed on the specification but does not exclude the existences or additional possibilities of other features, numbers, steps, operations, components, parts or combinations thereof in advance.

Otherwise indicated herein, all the terms used herein, which include technical or scientific terms, may have the same meaning that is generally understood by a person skilled in the art. In general, the terms defined in the dictionary should be considered to have the same meaning as the contextual meaning of the related art, and, unless clearly defined herein, should not be understood abnormally or as having an excessively formal meaning.

Hereinafter, preferred embodiments of the present invention will be described in more detail with reference to the accompanying drawings.

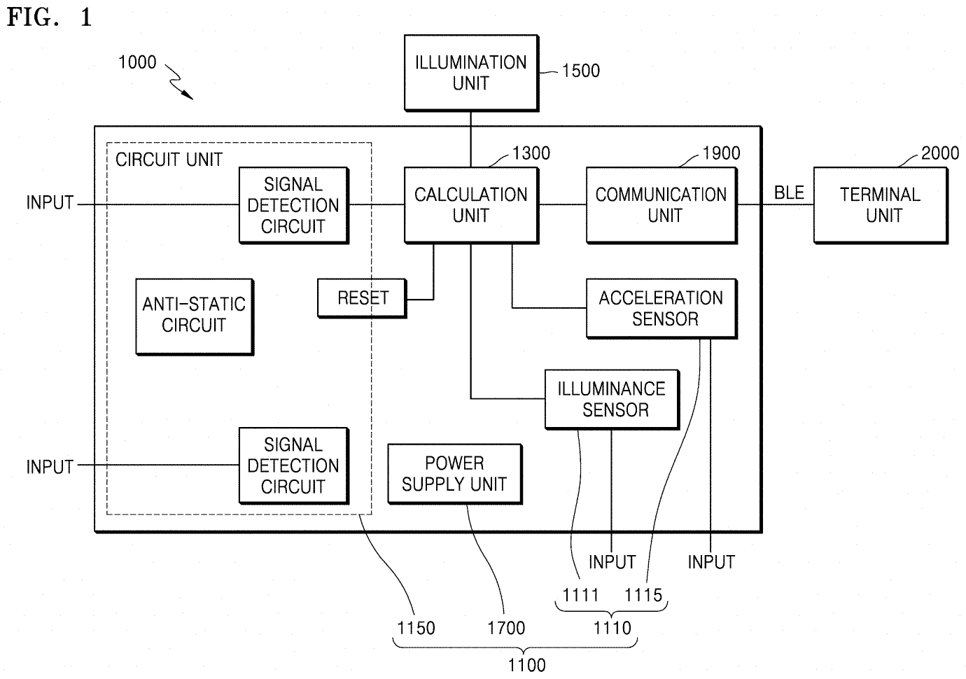

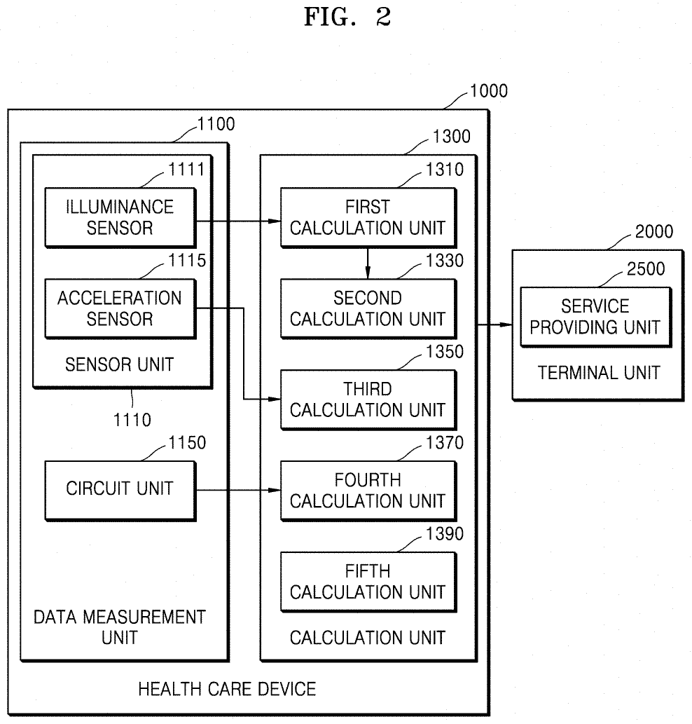

FIGS. 1 and 2 are configuration diagrams for explaining a health care device according to an embodiment of the present invention. More specifically, FIG. 1 is a configuration diagram for describing a health care device, and FIG. 2 is a configuration diagram for describing a data measurement unit and a calculation unit among health care devices.

Referring to FIGS. 1 and 2, a health care device 1000 according to an embodiment of the present invention may include a data measurement unit 1100, a calculation unit 1300, an illumination unit 1500, a power supply unit 1700, and a communication unit 1900.

The data measurement unit 1100 may measure basic data for extracting biometric data from a user. The data measurement unit 1100 may include a sensor unit 1110 and a circuit unit 1150.

The sensor unit 1110 may include an illuminance sensor 1111 and an acceleration sensor 1115. In addition, the sensor unit 1110 may further include a temperature sensor and an oxygen saturation measurement sensor. At this time, the temperature sensor and oxygen saturation measurement sensor can be used for the calculation of the stress index of the second calculation unit to be described later. A detailed description will be made during the description of the second calculation unit to be described later.

The illuminance sensor 1111 may measure basic data for calculating a user's heart rate and stress index together with the illumination unit 1500 to be described later.

The illuminance sensor 1111 may detect the amount of light transmitted from a user's body part. At this time, a part of the user's body may be a portion where blood flow changes due to a heartbeat appear. For example, the part measured by a body part may be a finger.

According to an embodiment, the illumination unit 1500 to be described later may irradiate light to a finger. Some of the irradiated light may be absorbed into the body by at least one of blood, bone, or tissue, and at least some of the light except for the absorbed light may pass through the finger.

At this time, the amount of light absorbed by the bone or tissue in the body may be small. In other words, the amount of absorption in the body of light by bone or tissue can be constant. On the other hand, the amount of absorption in the body of light by blood flow can be changed by heartbeat.

More specifically, arterial blood may flow through the capillaries of the fingers. At this time, the amount of blood in arterial blood can be changed by a heartbeat. Accordingly, the amount of transmission of light detected by the illuminance sensor 1111 may be provided in the form of a graph having a constant pattern. In other words, the graph of changes in blood flow may have a constant pattern shape.

The detected blood flow change data can be used as basic data for calculating a heart rate and a stress index calculated from a calculation unit to be described later.

The acceleration sensor 1115 may measure basic data for calculating a user's activity amount. According to an embodiment, the number of steps of the user may be measured by the acceleration sensor 1115. The method of extracting the user's activity amount from the acceleration sensor 1115 through the calculation unit 1300 will be described in more detail to be described later.

The circuit unit 1150 can measure electrical resistance. For example, the circuit unit 1150 may measure electrical resistance using a Bioelectrical Impedance Analysis (BIA) method.

The Bioelectrical Impedance Analysis (BIA) method is capable of conducting electric current in muscles and blood in which water is dissolved but may be a method of measuring electrical resistance using the principle that no current flows in the case of lean mass.

According to an embodiment, an electrical current may be measured by applying an AC current to at least part of the user's body. At this time, in order to prevent the discharge of the power supply unit 1700 to be described later, the circuit unit 1150 may block the applied AC current after the measurement of body composition is completed.

The electrical resistance measured by the circuit unit 1150 is transmitted to the fourth calculation unit 1370, which will be described later, and can be used as basic data for calculating body composition.

The calculation unit 1300 can extract biometric data from the basic data measured by the data measurement unit 1100. The calculation unit 1300 may include a first calculation unit 1310, a second calculation unit 1330, a third calculation unit 1350, and a fourth calculation unit 1370.

The first calculation unit 1310 may calculate heart rate data from basic data measured by the illuminance sensor 1111. More specifically, the first calculation unit 1310 may graph the amount of blood flow measured by the illuminance sensor 1111.

As described above, the graphed blood flow variation may exhibit a predetermined pattern. At this time, the predetermined pattern may mean a change in blood flow caused by a heartbeat. Therefore, it is possible to extract the heart rate data by calculating a period representing a predetermined pattern. For example, heart rate data can be measured by Photo PlethysmoGraph (PPG).

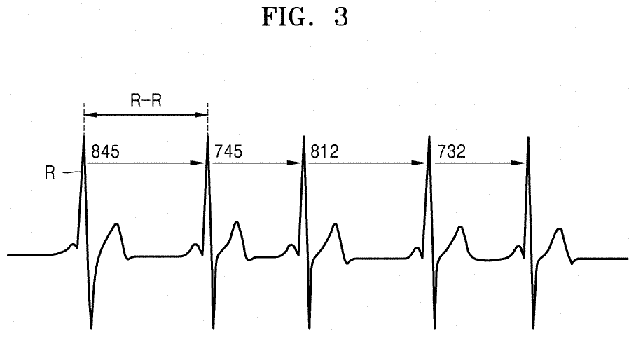

FIG. 3 is a graph for explaining a first calculation unit and a second calculation unit of a health care device according to an embodiment of the present invention.

Referring to FIGS. 1 to 3, the second calculation unit 1330 may calculate a stress index from the amount of blood flow graphed by the first calculation unit 1310.

In more detail, the stress index can be measured from a heart rate change (Heart RateVariability (HRV)) that is minutely changed by the autonomic nervous system.

The autonomic nervous system is divided into sympathetic and parasympathetic nerves, and the activity pattern may be different according to the user's psychological state.

The activities of the autonomic nervous system according to the user's psychological state are summarized in [Table 1] below.

TABLE 1

| | Sympathetic nerve | Parasympathetic nerve |
|---|---|---|
| Dominion | Extensive, high persistence | Local, low persistence |
| Physical difference | Excitement promotion | Excitement suppression |
| Psychological | Crisis situations (horror, | Peace, happiness, rest, |
| difference | anxiety, tension, exercise, | etc. |
| | etc.) | |
| Reaction rate | Delay occurrence of 5 | Sudden |
| | seconds or more | |

Referring to [Table 1], according to an embodiment, when the user is in a crisis situation such as fear, anxiety, and tension, or when an intense exercise is performed, the sympathetic nerve may be promoted.

According to another embodiment, when the user's psychology is stable, the parasympathetic nerve may be promoted.

Therefore, the user's stress index may be inferred from the sympathetic hyperactivity. In other words, the stress index can be calculated from the sympathetic hyperactivity.

The sympathetic nerve may have a faster heart rate during hyperactivity. Accordingly, in order to more clearly grasp the change in heart rate, the second calculation unit 1330 may secondarily differentiate the graph of blood flow change measured by the first calculation unit 1310.

Thereafter, the second calculation unit 1330 may calculate a stress index according to a change in the peak (R) interval (R-R) on the graph generated as a result of the second derivative.

In addition, when calculating the stress index of the second calculation unit 1330, the accuracy of the stress index can be increased by additionally reflecting oxygen saturation measurement data and temperature data. At this time, the oxygen saturation data and temperature data may be inputted from the outside or may be measured by the temperature sensor and oxygen saturation measurement sensor of the sensor unit 1110 described above.

The third calculation unit 1350 may calculate an activity amount from basic data measured by the acceleration sensor 1115.

More specifically, the third calculation unit 1350 may convert 3D coordinate data measured by the acceleration sensor 1115 into energy units by using a Signal Vector Magnitude (SVM).

The third calculation unit 1350 may set a threshold range of energy using Heuristic Algorithm (HA), and then measure the number of steps of the user.

Thereafter, the third calculation unit 1350 may calculate activity amount data through Adaptive Threshold Algorithm (ATA), which calculates the speed through the pace of the step.

The fourth calculation unit 1370 may be utilized as basic data for calculating body composition, as described above.

The fourth calculation unit 1370 may calculate the body composition using the electrical resistance value received from the circuit unit 1150.

As described above in the description of the circuit unit 1150, the magnitude of the electrical resistance value may mean the lean mass. Accordingly. The fourth calculation unit 1370 may convert the unit of the electrical resistance value to calculate the lean mass.

The fifth calculation unit 1390 may calculate obesity using the stress index calculated by the second calculation unit 1330, the activity amount calculated by the third calculation unit 1350, and the lean mass calculated by the fourth calculation unit 1370.

The following [Table 2] is a table summarizing the correlation of activity and body measurement based on the degree of stress index according to Pearson's product moment correlation coefficient P. Here, Pearson's product moment correlation coefficient may be a statistical coefficient describing the degree of linear relationship between variables as a value between 0 and 1.00. As the value of the correlation coefficient P is closer to 1, the correlation may be higher.

As shown in [Table 2] below, it can be seen that the stress index has a similar amount correlation with BMI, body fat percentage, abdominal fat percentage, and waist circumference.

TABLE

| | Small stress | | Medium stress | | Large stress | |
|---|---|---|---|---|---|---|
| | Man | Woman | Man | Woman | Man | Woman |
| Height (cm) | 0.56 | 0.055 | 0.041 | 0.095 | 0.057 | 0.074 |
| Weight (kg) | 0.155 | 0.139 | 0.122 | 0.137 | 0.187 | 0.166 |
| BMI (kg/m²) | 0.133 | 0.125 | 0.106 | 0.104 | 0.164 | 0.146 |
| Body fat | 0.119 | 0.150 | 0.107 | 0.111 | 0.159 | 0.162 |
| percentage | | | | | | |
| (%) | | | | | | |
| Abdominal fat | 0.120 | 0.145 | 0.101 | 0.096 | 0.159 | 0.146 |
| (cm) | | | | | | |
| Obesity (%) | 0.095 | 0.111 | 0.077 | 0.086 | 0.131 | 0.129 |
| Waist | 0.159 | 0.159 | 0.124 | 0.127 | 0.188 | 0.193 |
| circumference | | | | | | |
| (cm) | | | | | | |
| Activity | −0.092 | −0.111 | −0.076 | −0.093 | −0.095 | −0.074 |

(Source: A study on the correlation between life stress, body composition and body composition, serum lipids, and health-related behaviors of university students, Seong Min-jung (2008))

For example, when the stress index is high, large amounts of cortisol hormone may be produced. At this time, the cortisol hormone can interfere with the body's water metabolism and induce fat accumulation. Accordingly, the obesity rate may increase. Therefore, the fifth calculation unit 1390 can calculate the obesity degree by the stress index, the amount of activity, and the amount of lean body mass.

Referring back to FIG. 1, as described above, the illumination unit 1500 may irradiate a finger with light to calculate blood flow variation data. For example, the illumination unit 1500 can be an LED light, and can be provided as an integral or separate type.

The power supply unit 1700 may provide power to at least one of the data measurement unit 1100, the calculation unit 1300, the illumination unit 1500, or the communication unit 1700. In addition, the power supply unit 1700 may be charged by including a battery.

The communication unit 1900 may support a communication network with an external server. For example, the communication unit 1900 may provide a Bluetooth Low Energy 4.0 (BLE4.0) communication network.

According to an embodiment, the external server may be a terminal unit 2000 of a health care system to be described later. Biometric data measured by the calculation unit 1300 may be transmitted to the terminal unit 2000.

The health care device according to the embodiment of the present invention has been described above. A health care device includes a data measurement unit, a calculation unit, an illumination unit, a power supply unit, and a communication unit, and monitors the health status of the users by measuring biometric data of at least one of heart rate data, stress index, activity data, and body composition data from the user such that it can provide a high-efficiency and high-performance health care device capable of managing not only health but also a user's body shape by combining body composition data and a stress index.

Hereinafter, a health care system using the health care device will be described.

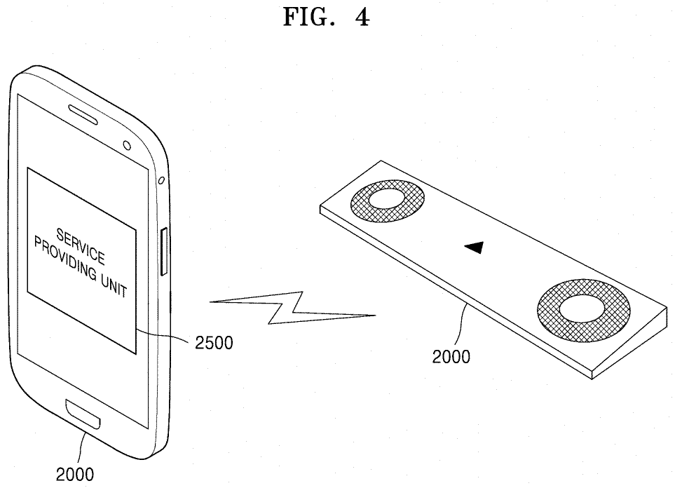

FIG. 4 is a perspective view for explaining a health care system according to an embodiment of the present invention.

Referring to FIGS. 1 to 4, the health care system may include a health care device 1000 and a terminal unit 2000.

The health care device 1000 is described redundantly by referring to FIGS. 1 to 2 in detail above and thus the description thereof will be omitted.

The terminal unit 2000 may receive biometric data from the communication unit 1700 of the health care device 1000. At this time, the terminal unit 2000 may be one of a mobile phone, a portable multimedia player (PMP), a mobile Internet device (MID), a smart phone, a desktop, a Tablet PC, a Note book, and a Net book, and other information communication devices.

The terminal unit 2000 may receive basic data from an external server. In this case, the basic data may include at least one of information input from a user or information input from an external server. For example, the basic data may be calories consumed, calorie reference table, and calorie consumption per exercise.

The terminal unit 2000 may include a service providing unit 2500 that processes biometric data calculated by the health care device 1000. The service providing unit 2500 may analyze and/or process at least one biometric data received from the health care device 1000.

The service providing unit 2500 may provide a customized health service to the user based on the analysis result.

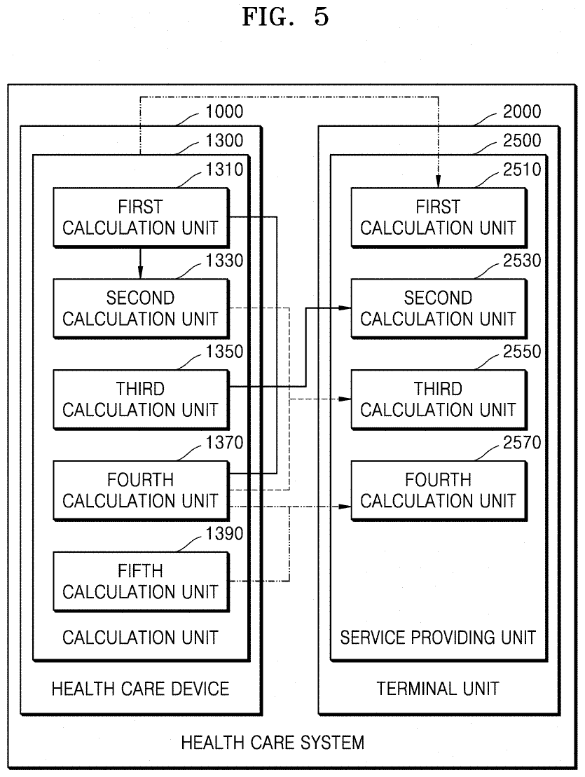

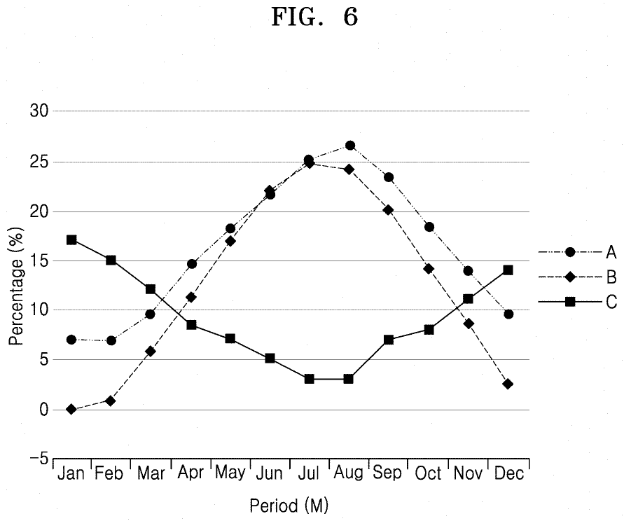

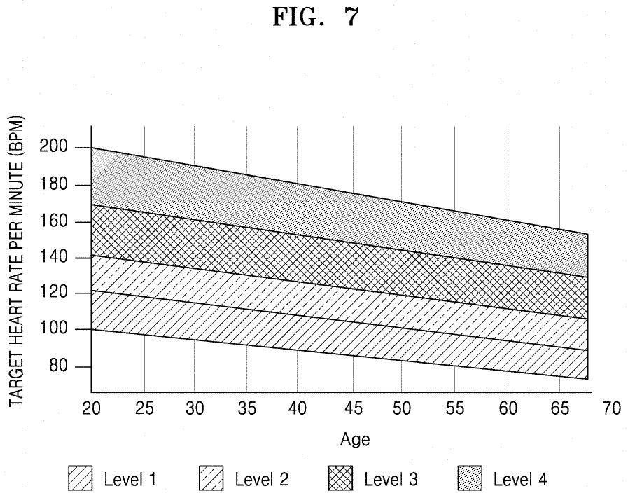

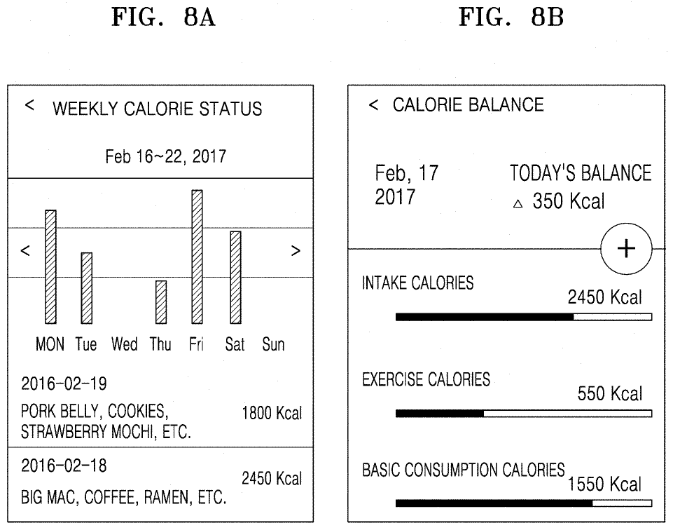

FIGS. 5 to 8 are configuration diagrams for describing service providing units according to an embodiment of the present invention. More specifically, FIG. 5 is a configuration diagram for explaining a service providing unit, FIG. 6 is a graph for explaining a first service providing unit, FIG. 7 is a graph for explaining a second service providing unit, and FIG. 8 is an image for explaining a third service providing unit.

Referring to FIGS. 1 to 8, the service providing unit 2500 includes a first service providing unit 2510, a second service providing unit 2530, a third service providing unit 2550, and a fourth service providing unit 20.

The first service providing unit 2510 may provide measurement information history management service. More specifically, the first service providing unit 2510 may collect biometric data calculated by the calculation unit 1100 of the health care device 1000. In other words, the first service providing unit 2510 may collect body fat data A, stress index B, and activity data C from the calculation unit 1100.

Subsequently, the first service providing unit 2510 may classify the biometric data A, B, and C for each measurement cycle, and display the trend graph form. At this time, the trend graphs for each data may individually control the display by the inquiry function.

The health care system according to an embodiment of the present invention provides a trend graph for each biometric data collected over a period of time by the first service providing unit 2510, so that changes in biometric data by period can be easily observed. Accordingly, it is possible to continuously monitor not only the amount of activity data that changes significantly in a short period of time, but also the change in body composition data and stress index gradually changing over the medium to long term.

The second service providing unit 2530 may provide a customized exercise recommendation service for each user.

More specifically, the second service providing unit 2530 may collect heart rate data, activity data, and body composition data from the calculation unit 1300 of the health care device 1000.

First, the second service providing unit 2530 may calculate a target heart rate per minute BPM for each exercise purpose based on the collected body composition data. At this time, the target heart rate per minute (BPM) may be a reference value for dividing the intensity of exercise intensity according to the exercise purpose.

More specifically, the target heart rate per minute (BPM) can be calculated by the karvonen formulas disclosed in [Equation 1] and [Equation 2] below (source, National Health Knowledge Center). [Equation 1] may be a formula applied when the user is a beginner of exercise, and [Equation 2] may be a formula applied when the user is an exercise expert.

BPM=(220−Age)*P [Equation 1]

BPM: heart rate per minute

Age: User's age

P: Exercise intensity (%)

BPM=((MAXBPM−NormalBPM)*P)+NormalBPM [Equation 2]

BPM: target heart rate per minute

MAXBPM: maximum heart rate

NormaIBPM: stable heart rate

P: Exercise intensity (%)

As in [Equation 1] and [Equation 2], different formulas may be applied to the target heart rate per minute (BPM) per exercise intensity according to the skill level of exercise. This is because the effect of exercise for each user may vary according to the skill level of exercise.

The skill level of the exercise may be determined according to the content of body composition for each user. For example, since there is more muscle power than a beginner of exercise in the case of an exercise expert, an exercise expert may have a lower exercise effect than a beginner who performs an exercise of the same intensity.

The following [Table 3] is a table summarizing exercise intensity and target heart rate per minute (BPM) by exercise purpose.

Referring to [Table 3] and FIG. 7, the target heart rate per minute BPM for each exercise purpose may vary according to the user's age.

TABLE 3

| | | | Target heart rate |
|---|---|---|---|
| Exercise purpose | Exercise intensity | Area (see FIG. 7) | (BPM, 20 years old) |
| Professional training | High intensity (85 ~ | Level 4 | 100~120 |
| | 100%) | | |
| Cardiopulmonary | Medium intensity (70 ~ | Level 3 | 120~140 |
| endurance | 85%) | | |
| improvement | | | |
| Weight loss | Low and medium | Level 2 | 140~170 |
| | intensity (60 ~ 70%) | | |
| Light exercise | Low intensity (50 ~ 60%) | Level 1 | 170~200 |

Subsequently, the second service providing unit 2530 may classify and store an exercise item suitable for an exercise purpose based on calorie consumption data for each exercise item provided from the outside. At this time, calorie consumption data for each sporting event may be provided from an external server.

The second service providing unit 2530 may recommend an exercise item stored in a corresponding area when an exercise purpose is selected by the user. In addition, when at least one exercise among the recommended exercise items is performed by a user, the second service providing unit 2530 may measure activity data from the time point at which the user's heart rate data measured by the calculation unit 1300 reaches the target heart rate per minute BPM.

The exercise recommendation service of the existing health care system did not reflect the error of exercise effect depending on the body composition content of each user. by simply performing a function that provides calorie consumption information for each event.

Moreover, the second service providing unit of the health care system according to the embodiment of the present invention recommends an exercise item optimized for an exercise purpose, and when the user's heart rate data in the exercise selected by the user satisfies the target heart rate per minute (BPM) range, it is possible to expect a maximized exercise effect that matches the exercise purpose by collecting and analyzing the amount of activity of the user.

The third service providing unit 2550 may provide a diet management service for each user.

More specifically, the third service providing unit 2550 may collect body composition data and activity data from the calculation unit 1300.

Thereafter, the third service providing unit 2550 may calculate the intake calorie amount from the collected data. For example, the intake calorie amount can be inputted from the user, and the intake calorie amount may be entered as calorie value data or as food name so that it can be stored as calorie information matching the calorie standard table.

After calculating the intake calorie amount, the third service providing unit 2550 may calculate the calorie amount consumed. The calorie amount consumed can be calculated by adding the amount of basic metabolism and exercise calories. For example, the basal metabolic rate can be transmitted from an external server, and the exercise calorie amount may be provided by processing the activity amount data calculated by the second service providing unit 2530.

The third service providing unit 2550 may calculate an intakeable calorie amount from the intake calorie amount and the calorie amount consumed.

The intakeable calorie amount can be calculated by subtracting the intake calorie amount from the sum of the recommended daily calorie amount and the calorie amount consumed. At this time, the daily recommended calorie amount may also be provided by an external server, and if the daily recommended calorie amount is negative, a warning sign may be transmitted to the user.

The third service providing unit 2550 may provide statues for each period of intake calorie amount, calorie amount consumed, and intakeable calorie amount in a graph form.

The fourth service providing unit 2570 may provide a body shape analysis service. More specifically, the fourth service providing unit 2570 may collect body composition data, stress index, activity data, and obesity from the calculation unit 1300.

Thereafter, the fourth service providing unit 2570 may calculate the daily intake limit calorie amount based on the collected data and the user's weight data.

The calculated daily intake calorie amount is updated at regular intervals to reflect the user's weight change, and is transmitted to the third service providing unit 2550 to be used in place of the recommended daily calorie.

The characteristics of the health care device and the health care system using the same have been described above. The health care device and the health care system using the same include a terminal unit including a health care device including a data measurement unit, a calculation unit, an illumination unit, a power supply unit, and a communication unit, and a service providing unit, so that precise biometric data measurement is possible, and based on measured biometric data, a high performance and high utilization health care device that provides services such as measurement information history management, exercise recommendation, diet management, and body analysis, and a health care system using the same may be provided.

While descriptions are made above with reference to the preferred embodiments of the present invention, it will be understood by those skilled in the art that various changes and modifications are possible without departing from the spirit and scope of the present invention as defined in the following claims.

1. A health care device comprising:
a data measurement unit configured to measure basic data from a sensor unit including an illuminance sensor and an acceleration sensor and a circuit unit measuring electrical resistance; and
a calculation unit configured to calculate biometric data of a user from the basic data.

2. The health care device of claim 1, further comprising an illumination unit for irradiating light to at least a portion of the user's body,
wherein the illuminance sensor measures an amount of transmission of light irradiated from the illumination unit to obtain blood flow change data.

3. The health care device of claim 2, wherein the calculation unit comprises:
a first calculation unit configured to calculate heart rate data from the blood flow change data obtained from the illuminance sensor;
a second calculation unit configured to calculate a stress index from the blood flow change data;
a third calculation unit configured to calculate activity data from walking data measured by the acceleration sensor; and
a fourth calculation unit configured to calculate a body composition from electrical resistance data measured by the circuit unit.

4. The health care device of claim 3, wherein the first calculation unit extracts a specific pattern from the blood flow change data and calculates a period for the pattern to measure heart rate data.

5. The health care device of claim 3, wherein the second calculation unit calculates a stress index according to a change in peak interval of a graph generated by secondary differentiation of the blood flow change data.

6. The health care device of claim 3, wherein the calculation unit further comprises a fifth calculation unit that calculates obesity by the stress index, the activity data, and the body fat mass.

7. A health care system comprising:
a health care device including a calculation unit for calculating biometric data of a user; and
a terminal including a service providing unit for analyzing the biometric data received from the health care device.

8. The health care system of claim 7, wherein the service providing unit comprises a first service providing unit for classifying at least one biometric data for each measurement session and displaying the classified biometric data in a trend graph form to provide a measurement information history management service.

9. The health care system of claim 8, wherein the trend graph is selectively displayed by a user.

10. The health care system of claim 7, wherein the service providing unit comprises a second service providing unit for calculating a target heart rate (Beats Per Minute (BPM)) using at least one of the biometric data and providing an exercise recommendation service.

11. The health care system of claim 10, wherein a calculation method for the target heart rate (Beats Per Minute (BPM)) varies according to a user's exercise skill.

12. The health care system of claim 11, wherein in the case of a beginner with low exercise skill, the target heart rate (Beats Per Minute (BPM)) is calculated by using a user's age and exercise intensity.

13. The health care system of claim 11, wherein in the case of an expert with high exercise skill, the target heart rate (Beats Per Minute (BPM)) is calculated by using a maximum heart rate, a stable heart rate, and an exercise intensity.

14. The health care system of claim 7, wherein the service providing unit comprises a third service providing unit for providing a diet management service by calculating an intakeable calorie amount from at least one of the biometric data.

15. The health care system of claim 7, wherein the service providing unit comprises a fourth service providing unit for providing a body type analysis service by calculating a recommended daily calorie amount from user's weight information and daily intake calorie amount.

16. The health care system of claim 7, wherein at least one biometric data measured by the health care device is transmitted to the terminal by Bluetooth communication.
